(19) United States
(12) Patent Application Publication
(10) Pub. No.: US 2006/0009373 A1
(43) Pub. Date: Jan. 12, 2006

(54) Method for treating diabetic wounds

(76) Inventor: Max Martin, Yukon, OK (US)

Correspondence Address: JAMES T. ROBINSON, 222 E. MAIN STREET, NORMAN, OK, 73069, US

(21) Appl. No.: 10/887,070

(22) Filed: Jul. 8, 2004

(51) Int. Cl. A61K 38/28; A61K 38/22

(52) U.S. Cl. 514/2; 514/3

(57) ABSTRACT

A method for treating diabetic wounds uses a topical preparation containing insulin in combination with glucose. The exact proportions are not critical, as the presence of even a trace amount of either component in combination with the other is effective. In one embodiment, the composition contains from about 0.1% to about 5.0% weight percent insulin in a 50 weight percent solution of glucose. In another embodiment, the topical preparation includes up to 1.0 weight percent silver sulfadiazine. In another embodiment, the topical preparation includes up to 100 parts per million by weight colloidal silver or ionic silver.

### BACKGROUND OF THE INVENTION

1. Field of the Invention

This invention relates to methods and compositions for promoting healing of wounds, especially diabetic wounds. More particularly, but not by way of limitation, the invention provides a combination of glucose and insulin for topical application. It is anticipated that the invention will also promote the healing of scrapes, burns, and external diabetic ulcers.

2. Discussion

There are numerous methods and compositions available to treat wounds and to promote wound healing. A wound may involve a laceration, cut or scrape, surgical incision, sore, thermal burn, puncture, or decubitus ulcer, e.g., bed sores. Partial thickness wounds involve damage to the epidermis and superficial dermis with no damage to the dermal blood vessels. Full thickness wounds involve loss of the dermis, extend to deeper tissue layers, and involve disruption of the dermal blood vessels. The healing of the partial thickness wound occurs by simple regeneration of epithelial tissue. Wound healing in full thickness wounds is more complex. The first stage involves an immediate inflammatory response, followed by a proliferative phase wherein no blood vessels (angiogenesis) and collagen are generated in the wound bed, resulting in the formation of granulation tissue. Contraction and re-epithelialization of the wound also occur during this phase. Finally, a maturation phase occurs during which collagen in the wound is subject to turnover and remodeling, ultimately resulting in the formation of scar tissue.

Intact epidermis serves as a mechanical barrier to infection. Due to the extensive tissue loss or damage in full thickness wounds, infection is more likely in full thickness wounds than in partial thickness injuries. Unless properly cleaned and treated, a full thickness wound may, in severe cases, result in the development of a life-threatening infection.

A large number of dressings, bandages, and topical preparations are available for the treatment of wounds. Passive wound dressings (e.g., gauze and adhesive bandages) serve only to provide mechanical protection and a barrier to infection. The dressings themselves do not supply any composition which enables or facilitates the healing process of the wound. Active dressings supply some biologically active compound to the site of a wound. One type of active dressing is a dressing or wrapping which delivers or has been impregnated with antimicrobials (e.g., Bacitracin).

In some cases, abnormal wound healing represents a significant health risk to patients. In particular, diabetic patients often experience slow or incomplete wound healing that may result in other serious consequences. Both type-I and type-II DM produce a variety of debilitating and life threatening complications, including degeneration of large and small blood vessels and increased susceptibility to infection. These two complications, unfortunately, make the DM patient prone to poor wound healing and wound infections. In extreme cases, limb amputation is necessary due to circulatory problems and infection at the site of wounds.

The present invention, consisting of insulin in combination with glucose, is effective in helping to promote wound healing in diabetic patients.

3. Summary of the Invention

The present invention provides a method for treating diabetic wounds with a topical preparation containing insulin in combination with glucose. The exact proportions are not critical, as the presence of even a trace amount of either component in combination with the other is effective. In one embodiment, the composition contains from about 0.1% to about 5.0% weight percent insulin in D50 (a 50% by weight aqueous solution of glucose). In another method according to the present invention, the insulin-glucose composition also includes up to 1% by weight silver sulfadiazine. In another method according to the present invention, the insulin-glucose composition also includes small quantities (less than 100 parts per million) of ionic or colloidal silver. In another method according to the present invention, Vitamin C is administered orally to the diabetic wound patient while the glucose-insulin composition is applied topically to the wound.

It is an objective of this invention to promote the healing of diabetic wounds using a topical application of a composition containing insulin and glucose.

Another object of the present invention is to promote the healing of scrapes, surgical incisions, sores, thermal burns, punctures, or bed sores by topical application of a composition containing insulin and glucose.

Other objects, features, and advantages of the present invention will become clear from the following description of the invention when read in conjunction with the appended claims.

### DETAILED DESCRIPTION OF THE INVENTION

This invention is based upon the finding that a composition containing insulin and glucose is useful for the treatment of diabetic and other wounds. As used herein, the term “insulin” encompasses both rapid-acting insulins, such as regular insulin and Semilente, which (if injected subcutaneously) start to work in 30-60 minutes and go on working for 5-16 hours, and long-acting insulins, such as Ultralente, which (if injected subcutaneously) start to work in 4-8 hours and continue working for 36 hours.

The composition containing both insulin and glucose, when applied topically to a diabetic wound, performs better than either insulin alone or glucose alone—even if the less concentrated component is present in only trace quantities.

The glucose included in the composition according to the present invention is more than a carrier. Glucose provides a key component for cell regeneration and prevents the dressing from adhering to the affected skin.

While the mechanism by which insulin promotes healing of the diabetic wound is unclear, it is believed that the insulin facilitates the uptake of the glucose by the cell, thereby speeding cell regeneration as compared to application of glucose alone to the affected area.

Silver sulfadiazine, a sulfa drug, is commonly used to prevent and treat infections of second-degree and third-degree burns. Silver sulfadiazine kills a wide variety of bacteria and is sometimes prescribed for other uses. Silver sulfadiazine normally comes in a cream (1% silver sulfadiazine by weight) for application once or twice daily to the affected area. Silver sulfadiazine is sold as a topical preparation in the United States under the brand names Silvadene, SSD, SSD AF, and Thermazene. In Canada, a silver sulfadiazine topical preparation is sold under the brand name Flamazine.

Homeopathic and naturopathic approaches to medicine have identified colloidal silver and ionic silver as potentially effective agents for treating bacterial, fungal, and viral infections. While the precise function of colloidal silver and ionic silver is unclear, it is believed that colloidal silver may fight infection and increase healthy cell reproduction.

Many preferred embodiments of this invention contain at least one or two, and sometimes several, other active ingredients in addition to insulin in combination with glucose.

Silver sulfadiazine may be added to the insulin-glucose composition in some embodiments. Effective treatment levels of up to 1% by weight should further promote wound healing.

Colloidal silver and ionic silver may be added to the insulin-glucose composition in low levels of up to 100 parts per million (0.01% by weight).

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

I claim:

1. A method for the treatment of diabetic wounds in patients, comprising:
applying a composition containing insulin and glucose to affected tissue.

2. A method in accordance with claim 1, wherein said insulin is about 0.1 percent to about 5.0 percent by weight of said composition.

3. A method in accordance with claim 1, wherein said insulin is about 0.5 percent to about 1.5 percent by weight of said composition.

4. A method in accordance with claim 1, wherein said composition further contains silver sulfadiazine.

5. A method in accordance with claim 4, wherein said silver sulfadiazine is 0.1 percent to about 1.0 percent by weight of said composition.

6. A method in accordance with claim 1, wherein said composition further contains colloidal silver.

7. A method in accordance with claim 6, wherein said colloidal silver is about 10 parts per million to about 100 parts per million by weight of said composition.

8. A method in accordance with claim 6, wherein said colloidal silver is about 1 part per million to about 10 parts per million by weight of said composition.

9. A method in accordance with claim 1, wherein said composition further contains ionic silver.

10. A method in accordance with claim 6, wherein said ionic silver is about 10 parts per million to about 100 parts per million by weight of said composition.

11. A method in accordance with claim 6, wherein said ionic silver is about 1 part per million to about 10 parts per million by weight of said composition.

12. A method for the treatment of skin damage in patients, comprising:
applying a composition containing insulin and glucose to affected tissue.

13. A method in accordance with claim 12, wherein said insulin is about 0.1 percent to about 5.0 percent by weight of said composition.

14. A method in accordance with claim 12, wherein said insulin is about 0.5 percent to about 1.5 percent by weight of said composition.

15. A method in accordance with claim 12, wherein said composition further contains silver sulfadiazine.

16. A method in accordance with claim 15, wherein said silver sulfadiazine is 0.1 percent to about 1.0 percent by weight of said composition.

17. A method in accordance with claim 12, wherein said composition further contains colloidal silver.

18. A method in accordance with claim 17, wherein said colloidal silver is about 10 parts per million to about 100 parts per million by weight of said composition.

19. A method in accordance with claim 17, wherein said colloidal silver is about 1 part per million to about 10 parts per million by weight of said composition.

20. A method in accordance with claim 12, wherein said composition further contains ionic silver.

21. A method in accordance with claim 20, wherein said ionic silver is about 10 parts per million to about 100 parts per million by weight of said composition.

22. A method in accordance with claim 20, wherein said ionic silver is about 1 part per million to about 10 parts per million by weight of said composition.

23. A method in accordance with claim 1, wherein said method further includes daily oral administration of Vitamin C to the patient.

24. A method in accordance with claim 2, wherein said method further includes daily oral administration of Vitamin C to the patient.
